(19) United States
(12) Patent Application Publication
(10) Pub. No.: US 2017/0304573 A1
(43) Pub. Date: Oct. 26, 2017

(54) MASK VENTILATION ASSIST DEVICE

(71) Applicant: Gerald P. Rosen, Miami Beach, FL (US)

(72) Inventor: Gerald P. Rosen, Miami Beach, FL (US)

(21) Appl. No.: 15/494,824

(22) Filed: Apr. 24, 2017

Related U.S. Application Data
(60) Provisional application No. 62/326,940, filed on Apr. 25, 2016.

(51) Int. Cl. A61M 16/06

(52) U.S. Cl. CPC A61M 16/0605; A61M 2205/586; A61M 2209/04

(57) ABSTRACT

A mask ventilation assist device that is attached to a mask to aid in ventilating a patient. Generally, the device comprises an opening that fits over the universal ventilation port of a mask, a cavity that compatibly mates with the mask, and one or more grips that permit a practitioner to apply downward force to the mask via the device. The device may comprise various shapes, sizes, configurations, and features depending on the type of mask on which the device will be deployed.

### CROSS REFERENCE TO RELATED APPLICATION

This application claims priority to, the benefit under 35 U.S.C. §119 of, and incorporates by reference herein in its entirety U.S. Provisional Patent Application No. 62/326,940, filed Apr. 25, 2016, and entitled “Mask Ventilation Assist Device.”

### TECHNICAL FIELD

The present disclosure relates generally to a mask ventilation assist device and, more particularly, to a device that is attached to a mask to aid in ventilating a patient.

### BACKGROUND

To prevent brain injury, cardiac arrest, and/or death, practitioners (e.g., nurses, doctors, emergency medical technicians, etc.) use masks to ventilate patients that are not breathing or that are not breathing sufficiently (preventing hypoventilation that could lead to both hypoxemia and acidosis). Generally, these masks come in only a few different sizes, for use depending on the size and shape of a patient's head, and have no hand grips on which the practitioner may place his or her hands. Accordingly, as the mask is placed over a patient's mouth, it is difficult to ensure a proper seal between the mask and the patient's face that prevents air from escaping from underneath the edges of the mask. For example, a patient's anatomical challenges, such as an abnormally-sized/shaped head, face, neck, facial hair, etc., or a practitioner's skill level, hand size, or upper body strength may impact whether a proper seal is achieved between the mask and the patient's face. Without a proper seal, the practitioner is unable to properly ventilate a patient to provide for adequate airflow.

Currently, common protocol for ventilation requires two practitioners: one practitioner to hold the mask to the patient's face with both hands and focus entirely on maintaining a leak-proof mask seal and one practitioner to actuate ventilation through the mask by squeezing an air(oxygen) source. Two-handed ventilation, however, does not always ensure an adequate mask seal to maintain ventilation because failure to anteriorize the mandible while maintaining a good mask seal during ventilation may prevent inadequate ventilation by impeding elevation of soft tissue obstruction in the distal pharynx, which obstructs airflow into the lungs and allowing outflow of carbon dioxide.

Therefore, there is a long-felt but unresolved need for a device that aids in ventilating a patient such that a proper seal between the mask and the patient's face may be easily achieved, as well as proper positioning of the patient, during ventilation.

### BRIEF SUMMARY OF THE DISCLOSURE

Briefly described, and according to one embodiment, aspects of the present disclosure generally relate to a device that is attached to a mask to aid in ventilating a patient such that a proper seal between the mask and the patient's face may be easily achieved, as well as proper positioning of the patient, during ventilation.

In various embodiments, a mask may be any device placed over a patient's mouth and nose to ventilate the patient (e.g., a bag-valve-mask manufactured and/or sold by AMBU®, 3M®, VITAL SIGNS, INC., KING MEDICAL®, etc.), with the practitioner applying pressure to the top of the mask downwards towards the face of the patient to seal the mask to the patient's face (thereby preventing airflow intended to enter the patient's lungs from escaping around the unsealed edges of the mask). These masks, in various embodiments, generally come in different shapes and/or sizes to accommodate the various sizes, shapes, and facial features of the human head but lack handles on which a practitioner may place his or her hands. In one embodiment, an exemplary mask comprises a port (also referred to as a “ventilation port,” “inlet port,” “outlet port,” “universal ventilation port,” etc.) that permits the flow of air through the mask and is centrally attached to a concave chamber that is shaped to fit over a patient's face and mouth (e.g., oval, triangular, etc.). The edges of the concave chamber, in one embodiment, may comprise a rubber seal or other gasket designed to seal the mask to the patient's face when the mask is pressed to the patient's face. The port of the mask, in various embodiments, may be attached to a bag, tubing, or machine (or may be left unattached to accept a practitioner's mouth) that provides airflow into the mask. In various embodiments, the mask may comprise additional valves to regulate the airflow through the mask.

The mask ventilation assist device, in various embodiments, creates a better seal between the mask and the face of the patient to which the mask is applied by allowing the practitioner to assert additional downward force on the mask while anteriorizing the mandible. Generally, the device comprises an opening that accepts the universal ventilation port of the mask. In one embodiment, the device further comprises two grips extending from the opening on which a practitioner may apply downward force to generate the seal between the mask and the patient's face. In various embodiments, the opening and two grips of the device define a curved form that is generally shaped to accept the concave chamber of a mask. In one embodiment, the device may comprise only one grip that generally further comprises the opening such that a practitioner may apply downward force to the mask using only one hand. In various embodiments, the one or two grips may comprise ridges to prevent the practitioner's hand from slipping off of the device.

In various embodiments, to ventilate a patient with the mask ventilation assist device, a practitioner generally places the device over the universal ventilation port of the mask (prior to attaching the bag, tubing, machine, etc. to the port) such that the curved form of the device nests with the concave chamber of the mask. The practitioner may then generally place the concave chamber of the mask over the patient's mouth and nose. In one embodiment, the practitioner may place his or her fingers, palms, hands, or any portion thereof on the grip(s) of the device, applying downward pressure to the grip(s) such that the mask is pressed onto the patient's face, ensuring a proper seal between the mask and the patient's face (thereby preventing airflow intended to enter the patient's lungs from escaping around the unsealed edges of the mask). In one embodiment, the practitioner may, to further stabilize the mask and to optimize anterior displacement of the patient's jaw, place their other fingers, that are not applying force to the device, at the base of the patient's mandible (optionally applying upward force on the same). In one embodiment, the practitioner may use one hand on the device and the other hand to operate the inflatable bag (or machine) attached to the mask.

In one embodiment, a device for assisting a user in securing a ventilation mask to the face of a patient, comprising: an arcuate frame having a first end and a second end, the arcuate frame having a middle portion defining an opening shaped to accommodate a ventilation port of the ventilation mask, wherein the arcuate frame has an arcuate shape that generally corresponds to a respective shape of the ventilation mask to allow the arcuate frame to nest compatibly with the ventilation mask; wherein both the first end and the second end each comprise a generally planar fin whose plane is generally orthogonal to the axis of the opening, whereby the generally planar fins are positioned to accommodate the hands of the user to apply a force on both the device and the ventilation mask.

In one embodiment, a mask ventilation assist device, comprising: a first handle; and a second handle attached to the first handle at an attachment point, wherein the attachment point defines an opening that is shaped to accept a port of a ventilation mask and wherein the first handle, the second handle, and the attachment point are shaped to compatibly nest with the ventilation mask.

In one embodiment, a mask ventilation assist device, comprising: a frame comprising a first end and a second end, wherein the frame defines an opening that is shaped to accept a port of a ventilation mask and wherein the frame is shaped to compatibly nest with the ventilation mask; a first handle attached to the first end of the frame; and a second handle attached to the second end of the frame.

In one embodiment, a kit comprising a mask ventilation assist device and instructions for use of the mask ventilation assist device to ventilate a patient.

According to one aspect of the present disclosure, the device, wherein a top surface of the generally planar fins comprises a friction-creating structure. Furthermore, the device, wherein the friction-creating structure comprises one or more ridges. Moreover, the device, wherein the generally planar fins are concave. Further, the device, wherein the opening comprises chamfered edges to better nest with the ventilation port and/or the ventilation mask. Additionally, the device, wherein the device is manufactured from a medical-grade material. Also, the device, wherein the medical-grade material comprises a latex-free plastic.

According to one aspect of the present disclosure, the mask ventilation assist device, wherein a top surface of the first handle and the second handle comprises a friction-creating structure. Furthermore, the mask ventilation assist device, wherein the friction-creating structure comprises one or more ridges. Moreover, the mask ventilation assist device, wherein at least a portion of the first handle and the second handle is concave. Further, the mask ventilation assist device, wherein the first handle and the second handle comprise arcuate fins. Additionally, the mask ventilation assist device, wherein the first handle and the second handle further comprise arcuate frames, each having a first end and a second end, wherein the arcuate fins are attached to the first ends and wherein the second ends are attached to the attachment point. Also, the mask ventilation assist device, wherein the first handle and the second handle are positioned on opposite sides of the opening.

According to one aspect of the present disclosure, the mask ventilation assist device, wherein the first handle and the second handle are positioned on the same side of the opening. Furthermore, the mask ventilation assist device, wherein the opening comprises chamfered edges to better nest with the port and/or the ventilation mask. Moreover, the mask ventilation assist device, wherein the device is manufactured from a medical-grade material. Further, the mask ventilation assist device, wherein the medical-grade material comprises a latex-free plastic.

According to one aspect of the present disclosure, the mask ventilation assist device, wherein a top surface of the first handle and the second handle comprises a friction-creating structure. Additionally, the mask ventilation assist device, wherein the friction-creating structure comprises one or more ridges. Also, the mask ventilation assist device, wherein the first handle and the second handle are concave. Furthermore, the mask ventilation assist device, wherein the first handle and the second handle are arcuate fins. Moreover, the mask ventilation assist device, wherein the first handle and the second handle are positioned on opposite sides of the opening. Further, the mask ventilation assist device, wherein the first handle and the second handle are positioned on the same side of the opening.

According to one aspect of the present disclosure, the mask ventilation assist device, wherein the opening comprises chamfered edges to better nest with the port and/or the ventilation mask. Additionally, the mask ventilation assist device, wherein the device is manufactured from a medical-grade material. Also, the mask ventilation assist device, wherein the medical-grade material comprises a latex-free plastic.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1A is a perspective view of an exemplary mask ventilation assist device, according to one embodiment of the present disclosure;

FIG. 1B is a top view of an exemplary mask ventilation assist device, according to one embodiment of the present disclosure;

FIG. 1C is a bottom view of an exemplary mask ventilation assist device, according to one embodiment of the present disclosure;

FIG. 1D is a side view of an exemplary mask ventilation assist device, according to one embodiment of the present disclosure;

FIG. 1E is an end view of an exemplary mask ventilation assist device, according to one embodiment of the present disclosure;

FIG. 1F is a cross-sectional view of an exemplary mask ventilation assist device (from line 1F from FIG. 1B), according to one embodiment of the present disclosure;

FIG. 2A is a top view of an alternative exemplary mask ventilation assist device, according to one embodiment of the present disclosure;

FIG. 2B is a cross-sectional view of an alternative exemplary mask ventilation assist device (from line 2B from FIG. 2A), according to one embodiment of the present disclosure;

FIG. 2C is a magnified view of an alternative exemplary mask ventilation assist device (within line 2C from FIG. 2B), according to one embodiment of the present disclosure;

FIG. 3 is a perspective view of an exemplary mask ventilation assist device in use on a mask, according to one embodiment of the present disclosure; and

FIG. 4 is a cross-sectional view of an alternative exemplary mask ventilation assist device in use on a mask and in use by a practitioner, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

Aspects of the present disclosure generally relate to a device that is attached to a mask to aid in ventilating a patient such that a proper seal between the mask and the patient's face may be easily achieved, as well as proper positioning of the patient, during ventilation.

In various embodiments, a mask may be any device placed over a patient's mouth and nose to ventilate the patient (e.g., a bag-valve-mask manufactured and/or sold by AMBU®, 3M®, VITAL SIGNS, INC., KING MEDICAL®, etc.), with the practitioner applying pressure to the top of the mask downwards towards the face of the patient to seal the mask to the patient's face (thereby preventing airflow intended to enter the patient's lungs from escaping around the unsealed edges of the mask). These masks, in various embodiments, generally come in different shapes and/or sizes to accommodate the various sizes, shapes, and facial features of the human head but lack handles on which a practitioner may place his or her hands. In one embodiment, an exemplary mask comprises a port (also referred to as a “ventilation port,” “inlet port,” “outlet port,” “universal ventilation port,” etc.) that permits the flow of air through the mask and is centrally attached to a concave chamber that is shaped to fit over a patient's face and mouth (e.g., oval, triangular, etc.). The edges of the concave chamber, in one embodiment, may comprise a rubber seal or other gasket designed to seal the mask to the patient's face when the mask is pressed to the patient's face. The port of the mask, in various embodiments, may be attached to a bag, tubing, or machine (or may be left unattached to accept a practitioner's mouth) that provides airflow into the mask. In various embodiments, the mask may comprise additional valves to regulate the airflow through the mask.

The mask ventilation assist device, in various embodiments, creates a better seal between the mask and the face of the patient to which the mask is applied by allowing the practitioner to assert additional downward force on the mask while anteriorizing the mandible. Generally, the device comprises an opening that accepts the universal ventilation port of the mask. In one embodiment, the device further comprises two grips extending from the opening on which a practitioner may apply downward force to generate the seal between the mask and the patient's face. In various embodiments, the opening and two grips of the device define a curved form that is generally shaped to accept the concave chamber of a mask. In one embodiment, the device may comprise only one grip that generally further comprises the opening such that a practitioner may apply downward force to the mask using only one hand. In various embodiments, the one or two grips may comprise ridges to prevent the practitioner's hand from slipping off of the device.

In various embodiments, to ventilate a patient with the mask ventilation assist device, a practitioner generally places the device over the universal ventilation port of the mask (prior to attaching the bag, tubing, machine, etc. to the port) such that the curved form of the device nests with the concave chamber of the mask. The practitioner may then generally place the concave chamber of the mask over the patient's mouth and nose. In one embodiment, the practitioner may place his or her fingers, palms, hands, or any portion thereof on the grip(s) of the device, applying downward pressure to the grip(s) such that the mask is pressed onto the patient's face, ensuring a proper seal between the mask and the patient's face (thereby preventing airflow intended to enter the patient's lungs from escaping around the unsealed edges of the mask). In one embodiment, the practitioner may, to further stabilize the mask and to optimize anterior displacement of the patient's jaw, place their other fingers, that are not applying force to the device, at the base of the patient's mandible (optionally applying upward force on the same). In one embodiment, the practitioner may use one hand on the device and the other hand to operate the inflatable bag (or machine) attached to the mask.

### Exemplary Embodiments

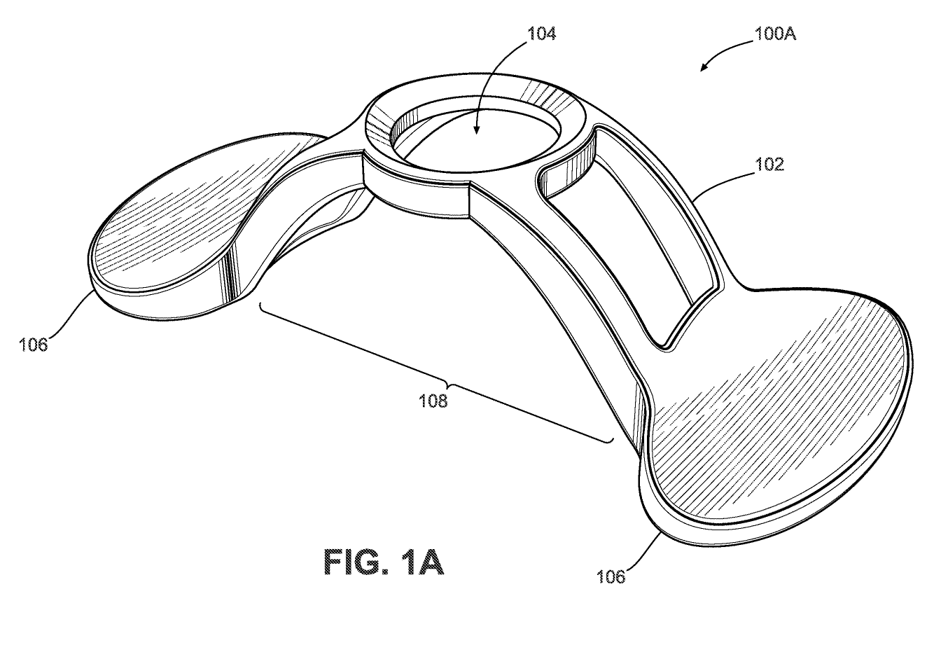

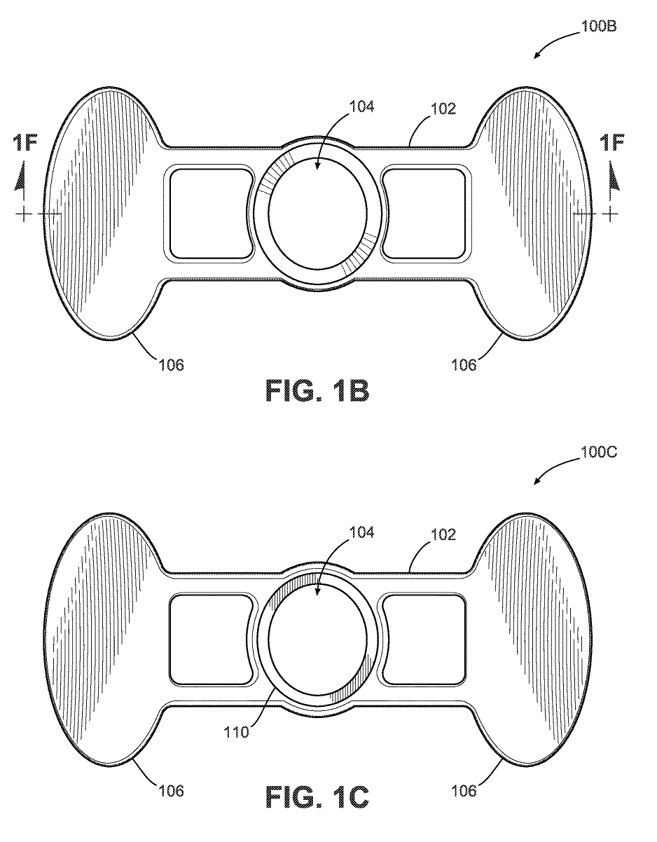

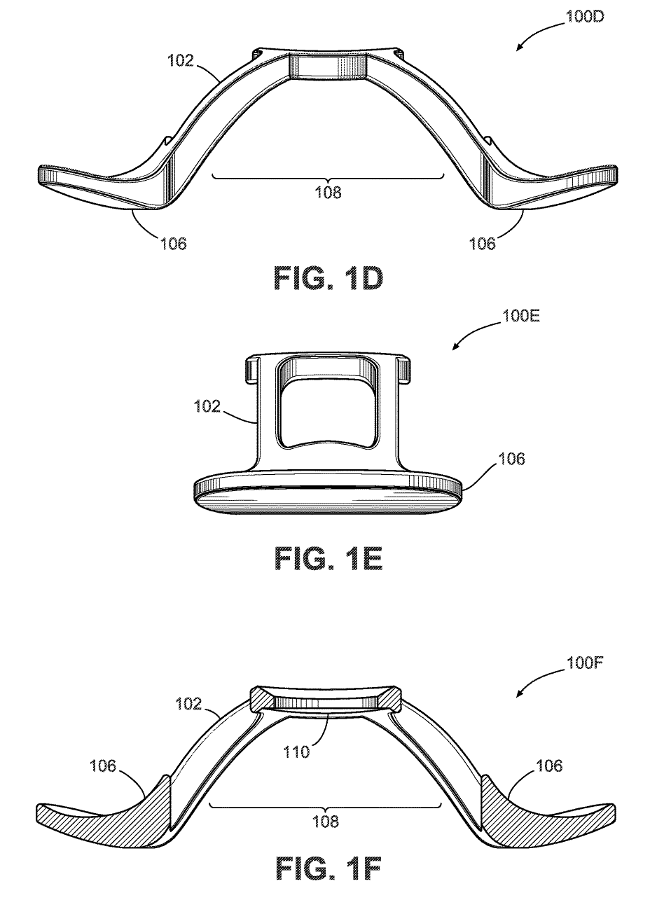

Referring now to the figures, for the purposes of example and explanation of the fundamental processes and components of the disclosed systems and methods, reference is made to FIG. 1 (consisting of FIGS. 1A-1F), which illustrates a perspective view 100A, top view 100B, bottom view 100C, side view 100D, end view 100E, and cross-sectional view 100F (from line 1F from FIG. 1B) of an exemplary mask ventilation assist device 102, according to one embodiment of the present disclosure. As will be understood and appreciated, the device 102 shown in FIG. 1 represents merely one approach or embodiment of the present disclosure, and other aspects are used according to various embodiments of the present disclosure. This disclosure generally places no limitations on the size or shape of the device 102 and the types of masks on which the device 102 can be deployed.

The device 102, in various embodiments, comprises an add-on to a mask (e.g., a bag-valve-mask manufactured and/or sold by AMBU®, 3M®, VITAL SIGNS, INC., KING MEDICAL®, etc.) that creates a better seal between the mask and the face of the patient to which the mask is applied by allowing a practitioner to assert additional force on the mask. Generally, the device 102 comprises an opening 104 that accepts the universal ventilation port of the mask, two grips 106 extending from the opening 104 on which a practitioner may apply downward force to generate the seal between the mask and the patient's face, and a cavity 108 defined by the curved form of the opening 104 and the two grips 106 that is generally shaped to accept the concave chamber of a mask.

In various embodiments, the opening 104 is a center, circular hole that fits tightly over the universal ventilation port of a mask. As will be understood by one having ordinary skill in the art, the size and shape of the opening 104 may depend on the mask on which the device 102 is to be deployed. For example, the bottom edge 110 of the opening 104, in one embodiment, may be adapted to accommodate and mate compatibility with any features of a particular mask. Further, in one embodiment, the opening 104 may be larger/smaller to accommodate a larger/smaller universal ventilation port. In one embodiment, the opening 104 is large enough to fit almost the entirety of a mask within the opening 104 (e.g., the device 102 is manufactured to rest on the edges of the mask).

The two grips 106 (alternatively referred to herein as “handles”), in various embodiments, extend downwards from the opening 104 and then terminate with a slight curve upwards (away from the cavity 108) so that a practitioner may apply force to both the device 102 and the mask. Generally, the practitioner applies downward force to the top surface of two grips using the palms of the practitioner's hands. In one embodiment, the two grips 106 are within the same vertical plane cutting through the device 102 such that the two grips 106 are on opposite sides of the opening 104 such that when a practitioner applies downward force to the two grips 106 any lateral force is cancelled out. In one embodiment, the two grips 106 are positioned on the same side of the opening 104 to magnify the lateral force created by the application of downward force to the two grips 106. In one embodiment, the device 102 may comprise only one grip 106 that generally further comprises the opening 104 such that a practitioner may apply downward force to the mask using only one hand. In one embodiment, the size and shape of the two grips 106 may be increased, thereby creating a larger surface area to which the practitioner may assert force.

In various embodiments, the cavity 108 defined by the curved form of the opening 104 and the two grips 106 is generally shaped to accept and mate with the concave chamber of a mask such that the device applies force to the entirety of the mask. Generally, the cavity 108 may be any shape or size depending on the type of mask on which it is to be deployed (e.g., smaller for a pediatric mask or a mask used with animals, larger/wider for a mask designed to fit over facial hair, etc.). In one embodiment, the device 102 does not have the cavity 108 and instead is manufactured to be flat (e.g., the two grips 106 and the opening 104 are within the same plane). The cavity 108 may, in one embodiment, be shaped to fit around a larger surface area of a mask (e.g., the device 102 may be wider than shown in FIG. 1) to generate better mask seal.

In one embodiment, the device 102 is manufactured as a part of or permanently affixed to a mask. In one embodiment, the device 102 is separate from a mask and is deployed on a mask for operation. In one embodiment, the device 102 is made from a medical-grade material (e.g., latex-free plastic, stainless steel, etc.).

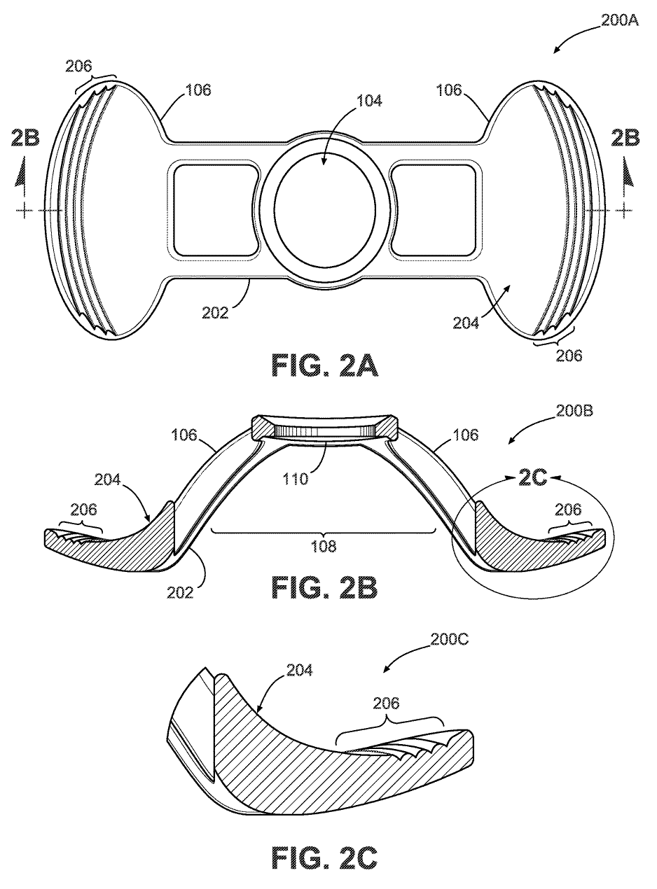

Now referring to FIG. 2 (consisting of FIGS. 2A-2C), a top view 200A, cross-sectional view 200B (from line 2B from FIG. 2A), and magnified view 200C (within line 2C from FIG. 2B) of an alternative exemplary mask ventilation assist device 202 is shown according to one embodiment of the present disclosure.

In various embodiments, the top surface 204 of the two grips 106 may comprise one or more ridges 206 to prevent the practitioner's hand from slipping off of the device 202. Generally, the ridges 206 may be any size, shape, height, width, or number so as to create enough friction to prevent the practitioner's hand from slipping off of the device 202 when downward force is applied to the device 202. For example, the top surface 204 may be covered in many narrow ridges 206 such that no portion of the top surface 204 is smooth. In an alternate embodiment, the top surface 204 may comprise only two or three deep, broad ridges 206. In various alternate embodiments, the one or more ridges 206 are bumps, sandpaper-like texture, rubber, or any other friction-creating structure/feature on the top surface 204.

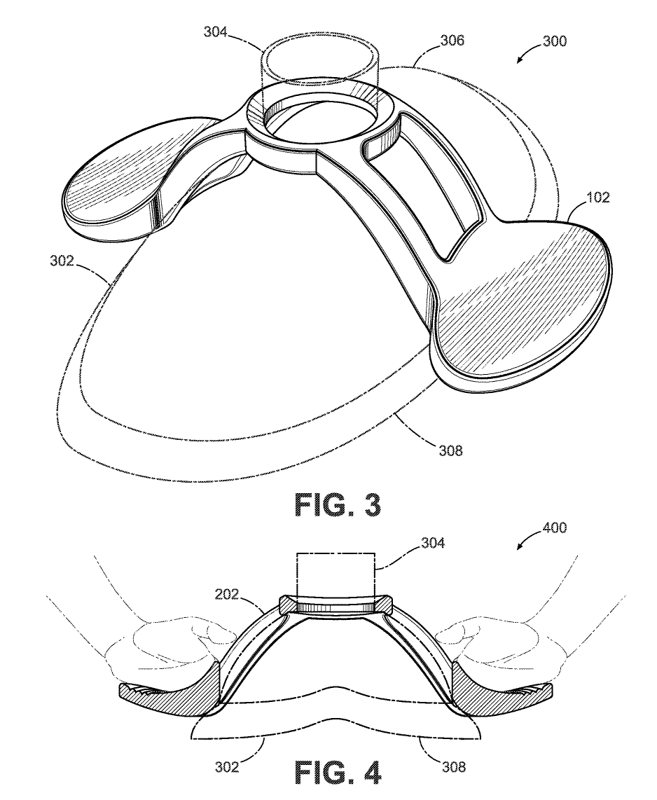

Referring now to FIG. 3, a perspective view 300 of an exemplary mask ventilation assist device 102 in use on a mask 302 is shown according to one embodiment of the present disclosure. These masks 302, in various embodiments, generally come in different shapes and/or sizes to accommodate the various sizes, shapes, and facial features of the human head but lack handles on which a practitioner may place his or her hands. In one embodiment, the mask 302 comprises a port 304 (also referred to as a “ventilation port,” “inlet port,” “outlet port,” “universal ventilation port,” etc.) that permits the flow of air through the mask 302 and is centrally attached to a concave chamber 306 that is shaped to fit over a patient's face and mouth (e.g., oval, triangular, etc.). The edges 308 of the concave chamber, in one embodiment, may comprise a rubber seal or other gasket designed to seal the mask to the patient's face when the mask is pressed to the patient's face.

In various embodiments, the device 102 is properly deployed on the mask 302 when the opening 104 of the device 102 accepts the universal ventilation port 304 of the mask 302. Generally, the universal ventilation port 304 is inserted up through the bottom of the opening 104 such that the cavity 108 of the device 102 fits over and compatibility mates with the concave chamber 306 of the mask 302. In one embodiment, all or a portion of the device 102 rests on the edge 308 of the mask 302. In one embodiment, the opening 104 is large enough to accept not only the universal ventilation port 304 but also a portion of the concave chamber 306 of the mask 302.

Now referring to FIG. 4, a cross-sectional view 400 of an alternative exemplary mask ventilation assist device 202 in use on a mask 302 and in use by a practitioner 402 (e.g., nurses, physicians, respiratory therapists, anesthesiologists, veterinarians, etc.) is shown according to one embodiment of the present disclosure.

Generally, masks 302 permit gas (e.g., air, oxygen, etc.) to be force-fed to a patient's trachea, bronchus, and lungs. For example, manual resuscitators (e.g., bag-valve masks) cause the gas inside an inflatable bag portion of the mask 302, which is attached to the universal ventilation port 304, to be force-fed to a patient via a one-way valve when the bag is compressed by the practitioner 402. To be effective in humans, a mask 302, in various embodiments, should deliver between about 500 and 800 milliliters of air to a normal male adult patient's lungs. In one embodiment, if supplemental oxygen is provided to the patient, 400 milliliters of air may still be adequate. In various embodiments, squeezing the bag once every 5 to 6 seconds for an adult or once every 3 seconds for an infant or child provides an adequate respiratory rate for a human (e.g., 10-12 respirations per minute in an adult and 20 per minute in a child or infant). Generally, to be effective, the practitioner 402 should ensure proper mask seal between the mask 302 and the patient's face. In one embodiment, proper mask seal generally creates an airtight or near-airtight seal between the edge 308 of the mask 302 and the face of the patient, which provides adequate air pressure needed to force-inflate the lungs and prevents release of air into the environment around the edges 308 of the mask 302.

In various embodiments, the practitioner 402 installs the device 202 (or 102 from FIG. 1) onto the mask by inserting the universal port 304 through the bottom of the opening 104 such that the cavity 108 of the device 202 fits over and compatibility mates with the concave chamber 306 of the mask 302. After ensuring the device 202 is properly installed, the practitioner 402 may, in one embodiment, attach the mask 302, via the universal port 304, to a bag, machine, or other apparatus that will generate airflow through the mask 302. In various embodiments, the practitioner 402 positions the mask 302 over the patient's nose and mouth. The practitioner 402, in various embodiments, generates a proper mask seal by placing his or her fingers, thumbs, palmer eminences, or other portion of his or her hands onto the grips 106 of the device 202 and applying downward force. In one embodiment, the practitioner 402 may use his or her other fingers at the base of the patient's mandible to optimize anterior displacement of the patient's jaw, which will create a better mask seal and elevate soft tissue in posterior pharynx facilitating air exchange. In one embodiment, the practitioner 402 may use one hand on the device 202 and the other hand to operate the bag, machine, or other apparatus attached to the mask 302 via the universal port 304—ensuring the appropriate amount of air is delivered to the patient (e.g., via squeezing the bag at the correct intervals, adjusting the ventilation machine, etc.).

While various aspects have been described in the context of a preferred embodiment, additional aspects, features, and methodologies of the claimed inventions will be readily discernible from the description herein, by those of ordinary skill in the art. Many embodiments and adaptations of the disclosure and claimed inventions other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed inventions. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed inventions. In addition, some steps may be carried out simultaneously, contemporaneously, or in synchronization with other steps.

The embodiments were chosen and described in order to explain the principles of the claimed inventions and their practical application so as to enable others skilled in the art to utilize the inventions and various embodiments and with various modifications as are suited to the particular use contemplated. Alternative embodiments will become apparent to those skilled in the art to which the claimed inventions pertain without departing from their spirit and scope. Accordingly, the scope of the claimed inventions is defined by the appended claims rather than the foregoing description and the exemplary embodiments described therein.

What is claimed is:

1. A device for assisting a user in securing a ventilation mask to the face of a patient, comprising:
an arcuate frame having a first end and a second end, the arcuate frame having a middle portion defining an opening shaped to accommodate a ventilation port of the ventilation mask, wherein the arcuate frame has an arcuate shape that generally corresponds to a respective shape of the ventilation mask to allow the arcuate frame to nest compatibly with the ventilation mask;
wherein both the first end and the second end each comprise a generally planar fin whose plane is generally orthogonal to the axis of the opening, whereby the generally planar fins are positioned to accommodate the hands of the user to apply a force on both the device and the ventilation mask.

2. The device of claim 1, wherein a top surface of the generally planar fins comprises a friction-creating structure.

3. The device of claim 2, wherein the friction-creating structure comprises one or more ridges.

4. The device of claim 1, wherein the generally planar fins are concave.

5. The device of claim 1, wherein the opening comprises chamfered edges to better nest with the ventilation port and/or the ventilation mask.

6. The device of claim 1, wherein the device is manufactured from a medical-grade material.

7. The device of claim 6, wherein the medical-grade material comprises a latex-free plastic.

8. A mask ventilation assist device, comprising:
a first handle; and
a second handle attached to the first handle at an attachment point, wherein the attachment point defines an opening that is shaped to accept a port of a ventilation mask and wherein the first handle, the second handle, and the attachment point are shaped to compatibly nest with the ventilation mask.

9. The mask ventilation assist device of claim 8, wherein a top surface of the first handle and the second handle comprises a friction-creating structure.

10. The mask ventilation assist device of claim 9, wherein the friction-creating structure comprises one or more ridges.

11. The mask ventilation assist device of claim 8, wherein at least a portion of the first handle and the second handle is concave.

12. The mask ventilation assist device of claim 8, wherein the first handle and the second handle comprise arcuate fins.

13. The mask ventilation assist device of claim 12, wherein the first handle and the second handle further comprise arcuate frames, each having a first end and a second end, wherein the arcuate fins are attached to the first ends and wherein the second ends are attached to the attachment point.

14. The mask ventilation assist device of claim 8, wherein the first handle and the second handle are positioned on opposite sides of the opening.

15. The mask ventilation assist device of claim 8, wherein the first handle and the second handle are positioned on the same side of the opening.

16. The mask ventilation assist device of claim 8, wherein the opening comprises chamfered edges to better nest with the port and/or the ventilation mask.

17. The mask ventilation assist device of claim 8, wherein the device is manufactured from a medical-grade material.

18. The mask ventilation assist device of claim 17, wherein the medical-grade material comprises a latex-free plastic.

19. A mask ventilation assist device, comprising:
a frame comprising a first end and a second end, wherein the frame defines an opening that is shaped to accept a port of a ventilation mask and wherein the frame is shaped to compatibly nest with the ventilation mask;
a first handle attached to the first end of the frame; and
a second handle attached to the second end of the frame.

20. The mask ventilation assist device of claim 19, wherein a top surface of the first handle and the second handle comprises a friction-creating structure.

21. The mask ventilation assist device of claim 20, wherein the friction-creating structure comprises one or more ridges.

22. The mask ventilation assist device of claim 19, wherein the first handle and the second handle are concave.

23. The mask ventilation assist device of claim 19, wherein the first handle and the second handle are arcuate fins.

24. The mask ventilation assist device of claim 19, wherein the first handle and the second handle are positioned on opposite sides of the opening.

25. The mask ventilation assist device of claim 19, wherein the first handle and the second handle are positioned on the same side of the opening.

26. The mask ventilation assist device of claim 19, wherein the opening comprises chamfered edges to better nest with the port and/or the ventilation mask.

27. The mask ventilation assist device of claim 19, wherein the device is manufactured from a medical-grade material.

28. The mask ventilation assist device of claim 27, wherein the medical-grade material comprises a latex-free plastic.

29. A kit comprising a mask ventilation assist device and instructions for use of the mask ventilation assist device to ventilate a patient.
